SEQUENCE LISTING (3 sequences)

SEQ ID NO: 1 - DNA, 18 bp, Artificial Sequence
  Fully synthetic
agagtttgatcctggctc

SEQ ID NO: 2 - DNA, 19 bp, Artificial Sequence
  Fully synthetic
ggttaccttgttacgactt

SEQ ID NO: 3 - DNA, 1049 bp, Artificial Sequence
  Fully synthetic
agaacagatttgtgggattggcttaacctcgcggtttcgctgccctttgttctgtccattgtagcacgtgtgtagcccaggtcataaggggcatgatgatttgacgtcatccccaccttcaagggttgcgctcgttgcgggacttaacccaacatctcacgacacgagctgacgacaaccatgcaccacctgtcactctgcccccgaaggggacgtcctatctctaggattgtcagaggatgtcaagacctggtaaggttcttcgcgttgcttcgaattaaaccacatgctccaccgcttgtgcgggcccccgtcaattcctttgagtttcagtcttgcgaccgtactccccaggcggagtgcttaatgcgttagctgcagcactaaggggcggaaaccccctaacacttagcactcatcgtttacggcgtggactaccagggtatctaatcctgttcgctccccacgctttcgctcctcagcgtcagttacagaccagagagtcgccttcgccactggtgttcctccacatctctacgcatttcaccgctacacgtggaattccactctcctcttctgcactcaagttccccagtttccaatgaccctccccggttgagccgggggctttcacatcagacttaagaaaccgccctgcgagccctttacgcccaataattccggacacgcttgccacctacgtattaccgcggctgctggcacgtagtagccgtgccttttctgataaggtaccgtcaaaggtaccggcctattcgaacggtacttgttcttccctaacaacagagctttacgatccgaaaaccttcatcactcacgcggcgttgctccgtcagactttcgtccattgcggaagattccctactgctgcctcccgtaggagtctgggccgtgtctcagtcccagtgtggccgatcaccctctcaggtcggctacgcatcgttgccttggtgagccgttacctcaccaactagctaatgcgccgcgggtccatctgtaagtggtagccgaagccaccttttatgtttgaa

(19) United States
(12) Patent Application Publication
(10) Pub. No.: US 2022/0009852 A1
(43) Pub. Date: Jan. 13, 2022

(54) BACILLUS SUBTILIS BS40-4 STRAIN AND METHOD FOR COMPOSTING ORGANIC WASTES BY USING THE SAME

(71) Applicant: Hebei University of Science and Technology, Shijiazhuang (CN)

(72) Inventors: Zaixing LI, Shijiazhuang (CN); Jun MA, Shijiazhuang (CN); Xiaosha WU, Shijiazhuang (CN); Yali HUANG, Shijiazhuang (CN); Xuebin HAN, Shijiazhuang (CN); Xue QIN, Shijiazhuang (CN); Shuang LIU, Shijiazhuang (CN)

(21) Appl. No.: 17/349,960

(22) Filed: Jun. 17, 2021

(30) Foreign Application Priority Data
Jul. 9, 2020 (CN) 202010657940.3

(51) Int. Cl. C05F 17/20; C12N 1/20; C12N 11/10; C12N 11/14

(52) U.S. Cl. CPC C05F 17/20; C12N 1/205; C12R 2001/125; C12N 11/14; C12N 11/10

(57) ABSTRACT

A Bacillus subtilis BS40-4 strain is deposited in China General Microbiological Culture Collection Center (CGMCC) with an accession number CGMCC No. 19757.

### CROSS-REFERENCE TO RELATED APPLICATIONS

Pursuant to 35 U.S.C. § 119 and the Paris Convention Treaty, this application claims foreign priority to Chinese Patent Application No. 202010657940.3 filed Jul. 9, 2020, the contents of which, including any intervening amendments thereto, are incorporated herein by reference. Inquiries from the public to applicants or assignees concerning this document or the related applications should be directed to: Matthias Scholl P C., Attn.: Dr. Matthias Scholl Esq., 245 First Street, 18th Floor, Cambridge, Mass. 02142.

### BACKGROUND

The disclosure relates to the field of environmental microorganisms, and more particularly to a Bacillus subtilis BS40-4 strain and a method for composting organic wastes by using the same.

With the increasing development of livestock and poultry breeding industry, the livestock and poultry manure has become one of the environmental pollution sources. Therefore, the efficient treatment of livestock and poultry manure has become a key factor to solving the problem of organic wastes pollutions. The high-temperature composting process is one of the important approaches for the resource utilization of livestock manure. The composting process employs the metabolism of microorganisms under suitable conditions to realize the oxidation, mineralization and aromatization of organic matter. The livestock and poultry manure includes a large amount of macromolecular lignocellulose that is difficult to degrade. Therefore, the traditional composting process of livestock and poultry manure mainly focuses on the degree of degradation and maturity extent of celluloses. However, in addition to the large amount of lignocellulose that is difficult to degrade, the livestock and poultry manure also includes complex macromolecular substances such as starch, fat and protein that have not been decomposed and utilized. Moreover, the acid-base changes of the livestock and poultry manure are relatively large, and the salinity is high and the compositions are very complicated. So, the traditional aerobic composting technology cannot achieve efficient decomposition of livestock and poultry manures. In addition, the traditional aerobic microorganisms have a low decomposition temperature and a long decomposition cycle, and is prone to produce a large amount of leachate, to cause secondary pollution during the maturation process.

For organic wastes with complex compositions such as livestock and poultry manure, bacterial agents containing a plurality of strains are used to improve their decomposition effect. However, as different strains have different environmental tolerance and optimal living conditions, the bacterial agents compounded by a plurality of microorganisms have no ideal effect.

### SUMMARY

The existing conventional aerobic microorganisms have the problems of long decomposition cycle, low decomposition temperature, poor degradation efficiency, and proneness to secondary pollution for the decomposition of organic wastes such as livestock and poultry manures with complex compositions.

To solve these problems, the disclosure provides a Bacillus subtilis BS40-4 and applications thereof.

A Bacillus subtilis BS40-4 strain, with the accession number China General Microbiological Culture Collection Center (CGMCC) No. 19757, belongs to Bacillus subtilis. It was deposited in the China General Microbiological Culture Collection Center on Apr. 28, 2020. The deposit address is Institute of Microbiology, Chinese Academy of Sciences, No. 3, Yard 1, Beichen West Road, Chaoyang District, Beijing.

Compared with the related art, the Bacillus subtilis BS40-4 provided by the disclosure maintains growth at a high temperature of 110° C., grows over a wide range of suitable temperature, and maintains a high activity at 30° C. to 100° C. In addition, the Bacillus subtilis BS40-4 provided by the disclosure has a strong ability to resist external harmful factors (high temperature, strong acid, strong alkali, high salinity) or adverse stimuli, and can survive in a wide range of pH (4.0-12.0) and maintain a high activity.

In addition to the characteristics of growth activity in an extreme environment, the Bacillus subtilis BS40-4 provided by the disclosure has significant cellulase activity, protease activity, lipase activity and amylase activity, and can be widely used in aerobic composting with the agricultural organic wastes such as livestock and poultry manure and crop straws as the raw materials, to improve the efficiency of composting. Therefore, the Bacillus subtilis BS40-4 of the disclosure is used in the decomposition and fermentation of organic wastes such as livestock and poultry manure with complex compositions, which increases the compost temperature, prolongs the high temperature period of the fermentation, and shortens the fermentation cycle.

The disclosure further provides the applications of the Bacillus subtilis BS40-4 in the decomposition and fermentation of organic wastes.

The Bacillus subtilis BS40-4 has high cellulase activity, protease activity, lipase activity and amylase activity, increasing the degree of decomposition of organic wastes compost, without leachate leakage. For organic wastes with complex compositions, there is no need to mix multiple strains and consider the growth environment of multiple strains. One strain can achieve a good composting effect, with extremely high application value.

The disclosure further provides a solid bacterial agent, and the solid bacterial agent comprises an adsorption carrier and the Bacillus subtilis BS40-4 bacterial powder.

In a class of this embodiment, the adsorption carrier comprises soluble starch or calcium carbonate.

In a class of this embodiment, the Bacillus subtilis BS40-4 bacterial powder is obtained by spray drying the fermentation broth of the Bacillus subtilis BS40-4.

In a class of this embodiment, the number of viable Bacillus subtilis BS40-4 in the solid bacterial agent is 1×10⁸ CFU/g to 5×10¹⁰ CFU/g.

In a class of this embodiment, the solid bacterial agent further comprises an inorganic nutrient comprising N, P₂O₅ and K₂O in a mass ratio of 4-8:1-3:1-3, and the addition amount of the inorganic nutrient is 4 to 9 times the mass of a bacterial powder of the Bacillus subtilis BS40-4.

The disclosure further provides a liquid bacterial agent, and the liquid bacterial agent comprises a nutrient solution and the Bacillus subtilis BS40-4 bacterial cell.

In a class of this embodiment, the nutrient solution comprises an inorganic nutrient comprising N, P₂O₅ and K₂O in a mass ratio of 4-8:1-3:1-3, and the mass concentration of the inorganic nutrient is 10-20% in the liquid bacterial agent.

In a class of this embodiment, the Bacillus subtilis BS40-4 bacterial cell is obtained by filtration of the Bacillus subtilis BS40-4 fermentation broth through a plate-and-frame filter.

In a class of this embodiment, the number of viable Bacillus subtilis BS40-4 in the liquid bacterial agent is 1×10⁸ CFU/mL to 5×10¹⁰ CFU/mL.

The disclosure further provides a method for composting organic wastes by using the solid bacterial agent. Specifically, the method comprises pulverizing the organic wastes, hydrolyzing pulverized organic wastes at 70 to 100° C., and adding the solid bacterial agent accounting for 0.1-0.3 wt. % of the organic wastes to the pulverized organic wastes, and evenly mixing for fermentation.

In the method for composting organic wastes by using the solid microbial agent provided by the disclosure, the hydrolysis is carried out at 70-100° C. before adding the solid bacterial agent to the pulverized raw material, which can further shorten the decomposition time. The entire cycle time of decomposition is shortened to 10 days while maintaining a higher decomposition degree.

In a class of this embodiment, the water content in the organic wastes is 50 to 70 wt. %.

In a class of this embodiment, the hydrolysis time is 2 to 10 hours.

In a class of this embodiment, intermittent aeration is performed during the fermentation process, and the intermittent aeration method is to conduct continuous aeration for 30 to 120 minutes and then stop for 30 to 40 minutes; the aeration rate per cubic meter of the pulverized raw material is 50 to 200 L/min during the continuous aeration process.

The disclosure further provides a method for composting organic wastes by using the liquid bacterial agent. Specifically, the method comprises pulverizing the organic wastes, hydrolyzing pulverized organic wastes at 70 to 100° C., and adding the liquid bacterial agent accounting for 0.1-0.3 wt. % of the organic wastes to the pulverized organic wastes, and evenly mixing for fermentation.

In a class of this embodiment, the water content in the organic wastes is 50 to 70 wt. %.

In a class of this embodiment, the hydrolysis time is 2 to 10 hours.

In a class of this embodiment, intermittent aeration is performed during the fermentation process, and the intermittent aeration method comprises conducting continuous aeration for 30 to 120 minutes and then stopping the aeration for 30 to 40 minutes; the aeration rate per cubic meter of the pulverized raw material is 50 to 200 L/min during the continuous aeration process.

### BRIEF DESCRIPTION OF THE DRAWINGS

FIG. 1 shows a colony morphology of the strain BS40-4 in accordance with Example 1 of the disclosure; and

FIG. 2 shows a microscopic examination of the strain BS40-4 in accordance with Example 1 of the disclosure.

### DETAILED DESCRIPTION

To further illustrate the disclosure, embodiments detailing a Bacillus subtilis BS40-4 and applications thereof are described below. It should be noted that the following embodiments are intended to describe and not to limit the disclosure.

### Example 1

1.1 Isolation of Strains

A sample of cow dung-straw compost product was collected from a dairy farm in Shijiazhuang, Hebei Province. The sample was separated by LB medium (formula: 10 g/L tryptone, 5 g/L yeast extract, 10 g/L NaCl) by a dilution plating procedure, to obtain 45 strains, after 5 times of passage in the plate, 45 monoclonal strains with stable hereditary were obtained and stored at −80° C.

The separated 45 monoclonal strains were cultured with the cellulose Congo red medium (formula: 1.0 g of sodium nitrate, 1.2 g of disodium hydrogen phosphate, 0.9 g of potassium dihydrogen phosphate, 0.5 g of magnesium sulfate, 0.5 g of potassium chloride, 0.5 g of yeast extract powder, 0.5 g of acid hydrolyzed casein, 0.2 g of Congo red, 5.0 g of cellulose powder, 15.0 g of agar, 1000 mL of distilled water, pH 7.0±0.1, autoclaved at 121° C. for 15 min) in an incubator at 50° C. for 72 h. According to the transparent circle index of the culture medium colony (diameter of transparent circle D/colony diameter d≥4), the strains with high temperature resistance, cellulase secretion and high activity were isolated and screened, to obtain 21 strains with high temperature resistance and high activity of cellulose, which were stored at −80° C.

The 21 strains with high temperature resistance and high activity of cellulase obtained from the preliminary screening were taken out from −80° C., streaked on LB plates for activation, and cultured at 50° C. for 24 hours. The colonies on the LB plates were scraped off with an inoculating loop and inoculated in a conical flask containing 50 mL of LB broth medium, and cultivated on a shaker at 50° C. and 200 r/min for 24 h. 1 mL of the above culture solution was inoculated into a straw culture medium (formula: 2 g of crushed corn stalk, 3 g of urea, 6 g of (NH₄)₂SO₄, 3 g of peptone, 0.1 g of CaCl₂), 0.5 g of MgSO₄.7H₂O, 1 g of K₂HPO₄, 0.1 g of NaCl, 0.05 g of FeSO₄.7H₂O, 0.016 g of MnSO₄.7H₂O, 0.014 g of ZnSO₄.7H₂O, 0.037 g of CoCl₂.6H₂O, 1000 mL of distilled water, pH 7.0±0.1, autoclaved at 121° C. for 30 min), cultured in an incubator at 50° C. for 10 days, and the straw degradation was tested. The straw degradation rates of 6 strains with high temperature tolerance and high activity of cellulase were shown in Table 1. The strain BS40-4 with the best degradation of corn straw was stored at −80° C. The straw degradation rate (%)=(W₀−Wᵢ)/W₀×100%, W₀ represented the dry mass of the straw in the culture medium before the inoculation of the strain (g); Wᵢ represented the dry mass of the straw in the medium at the end of culture (g).

TABLE 1
Straw degradation rates of 6 strains with high temperature
tolerance and high activity of cellulose

| Strain | Straw degradation rate % |
|---|---|
| BS40-1 | 28.8 |
| BS40-2 | 30.2 |
| BS40-3 | 29.6 |
| BS40-4 | 36.6 |
| BS40-5 | 26.5 |
| BS40-6 | 27.3 |

1.2 Identification of 16S rRNA of Strain BS40-4.

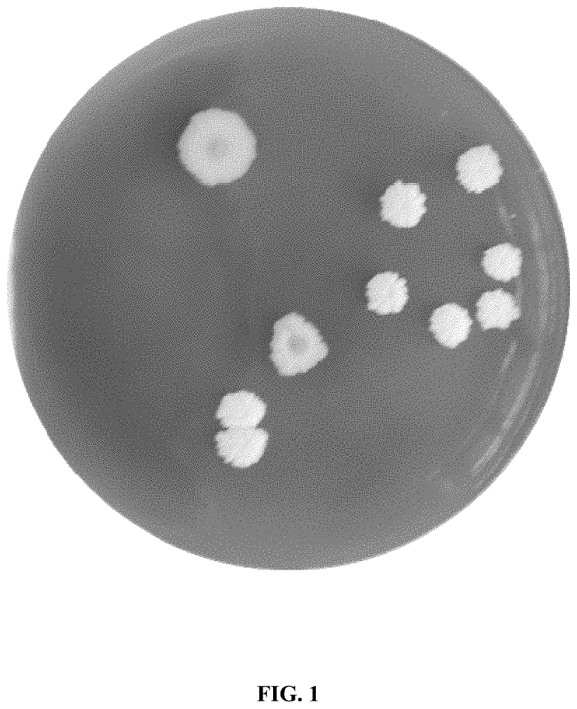

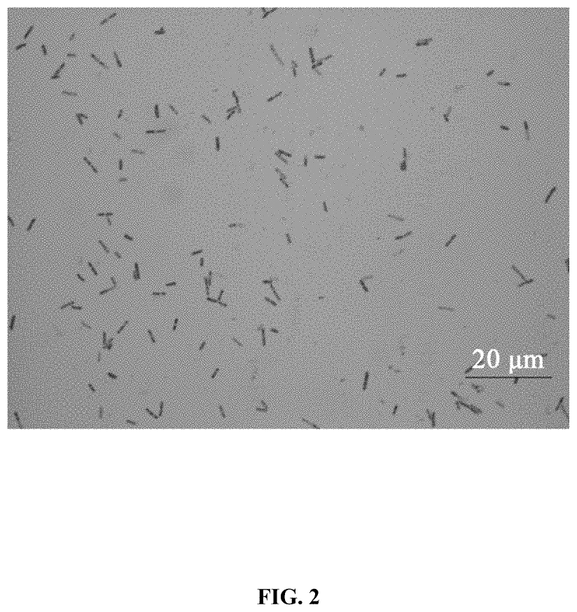

The BS40-4 strain stored at −80° C. was cultured on LB solid medium at 50° C. for 2 days. The observation on the colony morphology showed that the colony surface was rough and opaque, slightly white or slightly yellow, as shown in FIG. 1; the cell morphology was observed under a microscope, the cells were rod-shaped, with a diameter of 0.6 μm-1.0 μm, a length of 1.5 μm-2.0 μm, and central spore in an ellipse shape, as shown in FIG. 2.

The genomic DNA of the BS40-4 strain was extracted. Using the genomic DNA of the BS40-4 strain as a template to perform PCR amplification with the upstream primer 27f: 5′-AGAGTTTGATCCTGGCTC-3′ (SEQ ID NO: 1) and the downstream primer 1492r: 5′-GGTTACCTTGTTACGACTT-3′ (SEQ ID NO: 2) of the 16SrRNA universal primer. The PCR was in accordance with the following procedure: pre-denaturation at 95° C. for 5 min; denaturation at 95° C. for 30 s, annealing at 55° C. for 45 s, extension at 72° C. for 60 s, 30 cycles; and extension at 72° C. for 10 min. The purity and size of the amplified product were detected by electrophoresis, and the PCR product with the correct amplification length was sent to Shanghai Sangon for sequencing. The sequence was shown as SEQ ID NO: 3 and BLAST was performed in NCBI. According to the BLAST result, the similarity of 16SrRNA gene sequences between the strain BS40-4 and Bacillus subtilis was 100%.

The colony morphology is analyzed in combination with the 16SrRNA sequence. The result showed that the strain BS40-4 belonged to Bacillus subtilis and was named as Bacillus subtilis BS40-4. The strain was deposited in the China General Microbiological Culture Collection Center on Apr. 28, 2020 (address: Institute of Microbiology, Chinese Academy of Sciences, No. 3, Yard 1, Beichen West Road, Chaoyang District, Beijing), with the accession number CGMCC No. 19757.

1.3 Identification of the Physiological Characteristics of Bacillus subtilis BS40-4

Bacillus subtilis BS40-4 stored at −80° C. was streaked on LB plates and cultured at 50° C. for 24 h. The BS40-4 colonies on the LB plates were scraped off with an inoculating loop and inoculated in a conical flask containing 50 mL of LB broth medium, and cultivated on a shaker at 50° C. and 200 r/min for 24 h.

1 mL of the BS40-4 LB broth was taken and inoculated on the cellulose Congo red solid medium, and cultured at 20° C., 30° C., 50° C., 60° C., 75° C., 90° C., 100° C. and 110° C. for 3 days, respectively, to observe the growth conditions of strain BS40-4 under different temperature conditions. The results were shown in Table 2.

TABLE 2
Growth conditions of BS40-4 under different temperature conditions

| Temperature | 20° C. | 30° C. | 50° C. | 60° C. | 75° C. | 90° C. | 100° C. | 110° C. |
|---|---|---|---|---|---|---|---|---|
| Growth | + | ++ | +++ | +++ | +++ | ++ | ++ | + |
| conditions | | | | | | | | |

Note:
+ represented the strain could grow (colonies on solid medium), ++ represented that the strain grew well (the number of colonies on the solid medium was 10 to 50), +++ represented the strain grew vigorously (the number of colonies on solid medium was more than 50).

As shown from Table 2, Bacillus subtilis BS40-4 could grow well on the cellulose Congo red medium under high temperature conditions, belonging to a thermostable bacterium.

1 mL of the above BS40-4 LB broth was taken and inoculated on the cellulose Congo red solid medium with pH 4.5, 6.0, 7.5, 9.0, 10.5 and 11.5 respectively, and cultured at 50° C. for 3 days. The growth conditions of the strain BS40-4 under different temperature conditions were observed. The results were shown in Table 3.

TABLE 3
Growth conditions of BS40-4 at different pH values

| pH | 4.0 | 4.5 | 6.0 | 7.5 | 9.0 | 10.5 | 11.5 | 12.0 |
|---|---|---|---|---|---|---|---|---|
| Growth | + | ++ | ++ | +++ | +++ | +++ | ++ | + |
| conditions | | | | | | | | |

Note:
+ represented the strain could grow (colonies on solid medium), ++ represented that the strain grew well (the number of colonies on the solid medium was 10 to 50), +++ represented the strain grew vigorously (the number of colonies on solid medium was more than 50).

As shown from Table 3, Bacillus subtilis BS40-4 could grow well on the cellulose Congo red medium under extreme pH conditions, belonging to an acid and alkali-resistant bacterium.

1.4 Identification of Enzyme Producing Activity of Bacillus subtilis BS40-4

The lipase-producing ability of Bacillus subtilis BS40-4 strain was validated. The BS40-4 strain was activated and inoculated into a nutrient broth medium (10 g/L peptone, 3 g/L beef powder extract, 5 g/L sodium chloride, pH 7.2±0.2), and cultured at 50° C. for 24 h. The activity of lipase in the broth was determined by alkali titration method. After determination, the activity of lipase in the culture medium was 10.2 U/mL.

The activated strain BS40-4 was inoculated into a 50 mL of skimmed milk powder medium (10 g of peptone, 3 g of beef extract, 5 g of sodium chloride, 1.5 g of skimmed milk powder, 1000 mL of distilled water), and cultured at 50° C. for 24 h. The Folin-phenol method was used to determine the activity of protease in the mixture. After determination, the activity of the protease in the mixture was 20.7 U/mL.

The activated strain BS40-4 was inoculated into a starch culture solution (10 g of peptone, 5 g of beef extract, 5 g of sodium chloride, 2 g of soluble starch, 1000 mL of distilled water), and cultured at 50° C. for 24 h. The NDS method was used to determine the activity of amylase in the mixture. After determination, the activity of the amylase in the culture solution was 48.7 U/mL.

The activated strain BS40-4 was inoculated into a cellulose culture solution (10 g of peptone, 5 g of beef extract, 5 g of sodium chloride, 5 g of sodium carboxymethylcellulose, 1000 mL of distilled water), and cultured at 50° C. for 24 h. The CMC saccharification power method was used to determine the activity of cellulase in the mixture. After determination, the cellulase activity in the culture solution was 102.8 U/mL.

The identification of enzyme producing activity of Bacillus subtilis BS40-4 showed that, the Bacillus subtilis BS40-4 had the ability to produce lipase, protease, amylase and cellulose.

### Example 2

Preparation of Solid Bacterial Agent of Bacillus subtilis BS40-4.

For the Bacillus subtilis BS40-4 deposited in Example 1, the following steps were performed in sequence:

strain activation: the BS40-4 strain stored at −80° C. was streaked to inoculate on a LB plate, and inoculated at 50° C. for 24 h;

culture of primary seeds: the BS40-4 colonies on the LB plate were scrapped off and inoculated in a conical flask containing LB broth medium, and cultured at 50° C. and 200 r/min on a shaker for 24 h;

fermentation of secondary seeds: the above-mentioned primary seed culture solution was inoculated into a seed tank containing 10 L of LB broth medium at 10% inoculation amount, and fermented at 50° C. and 200 rpm/min for 1 day;

expanded culture: the resulting secondary seed fermentation broth was inoculated into a fermentor containing 50 L of LB broth medium at 5% inoculation amount, and fermented at 50° C. and 200 rpm/min for 2d; and

spray drying: spray drying of the expanded culture solution of Bacillus subtilis BS40-4 was performed to obtain the Bacillus subtilis BS40-4 bacterial powder.

The resulting Bacillus subtilis BS40-4 bacterial powder was mixed with the soluble starch to make the number of viable cells to reach 1×10⁹ CFU/g, then inorganic nutrients of 5 times the mass of the bacterial powder (N, P₂O₅ and K₂O at a ratio of 3:1:1) were added, and mixed well, to obtain a solid bacterial agent of Bacillus subtilis BS40-4.

### Example 3

Preparation of a Liquid Bacterial Agent of Bacillus subtilis BS40-4.

The expanded culture solution of Bacillus subtilis BS40-4 obtained in Example 2 was filtered by a plate and frame method to obtain Bacillus subtilis BS40-4 bacterial cells.

The resulting Bacillus subtilis BS40-4 bacterial cells were added to the nutrient solution (containing inorganic nutrients at a mass concentration of 10-20%, and the mass ratio of N, P₂O₅ and K₂O at 3:1:1 in the inorganic nutrients), to make the number of viable bacteria to reach 1×10⁹ CFU/mL, to obtain a liquid bacterial agent of Bacillus subtilis BS40-4.

### Example 4

The fresh pig manure and corn stalks were used as raw materials, and the solid bacterial agent in the Example 2 was used for composting. The specific method was as follows:

pretreatment of raw materials: fresh pig manure (73.5% moisture content) and straws (6.9% moisture content) were used; the straws were pulverized and crushed into particles with a particle size of ≤50 mm; the fresh pig manure was heated to 90° C. and maintained for 5 h, and then mixed with the crushed straws according to a mass ratio of 4:1 (pig manure:straw), and inoculated with a solid bacterial agent at the inoculation amount of 0.2% of the mass of the fermentation raw materials; the initial C/N ratio of the mixed raw materials was 25, and the moisture content was 60%; the mixture was subjected to intermittent aeration during the fermentation process, and the aeration volume per cubic meter of raw material was 100 L/min; after continuous aeration for 80 min, the aeration was stopped for 30 min.

During the fermentation process, the temperature reached 45° C. on the second day of fermentation. The high temperature period lasted 8 days, and the maximum temperature of the fermentation process reached 96° C.

After the end of the fermentation process, the fermentation product was subjected to microbial testing. The killing rates of both E. coli and Ascaris eggs reached 100%.

The fermentation product was in a loose state and did not produce any ammonia odor. The fermentation product has C/N ratio of 12.3, water content of 30%, seed germination rate of 99%, and no leachate was produced.

### Example 5

The fresh pig manure and corn stalks were used as raw materials, and the liquid bacterial agent in the Example 3 was used for composting. The specific method was as follows:

pretreatment of raw materials: fresh pig manure (73.5% moisture content) and straws (6.9% moisture content) were used; the straws were pulverized and crushed into particles with a particle size of ≤50 mm; the fresh pig manure was heated to 90° C. and maintained for 5 h, and then mixed with the crushed straws according to a mass ratio of 4:1 (pig manure:straw), and inoculated with a liquid bacterial agent at the inoculation amount of 0.2% of the mass of the fermentation raw materials; the initial C/N ratio of the mixed raw materials was 25, and the moisture content was 60%; the mixture was subjected to intermittent aeration during the fermentation process, and the aeration volume per cubic meter of raw material was 100 L/min; after continuous aeration for 80 min, the aeration was stopped for 30 min.

During the fermentation process, the temperature reached 45° C. on the second day of fermentation. The high temperature period lasted 8 days, and the maximum temperature of the fermentation process reached 96° C.

After the end of the fermentation process, the fermentation product was subjected to microbial testing. The killing rates of both E. coli and Ascaris eggs reached 100%.

The fermentation product was in a loose state and did not produce any ammonia odor. The fermentation product has C/N ratio of 12, water content of 20%, seed germination rate of 99%, and no leachate was produced.

It will be obvious to those skilled in the art that changes and modifications may be made, and therefore, the aim in the appended claims is to cover all such changes and modifications.

What is claimed is:

1. A Bacillus subtilis BS40-4 strain, being deposited in China General Microbiological Culture Collection Center (CGMCC) with an accession number CGMCC No. 19757.

2. A bacterial agent, comprising an auxiliary agent and the Bacillus subtilis BS40-4 strain of claim 1.

3. The bacterial agent of claim 2, wherein:
the bacterial agent is a solid bacterial agent;
the auxiliary agent comprises an adsorption carrier; and the adsorption carrier comprises soluble starch or calcium carbonate; and/or
the Bacillus subtilis BS40-4 strain is in the form of powders obtained by spray drying a fermentation broth of the Bacillus subtilis BS40-4 strain.

4. The bacterial agent of claim 3, wherein a number of viable Bacillus subtilis BS40-4 in the solid bacterial agent is 1×10⁸ CFU/g to 5×10¹⁰ CFU/g.

5. The bacterial agent of claim 3, wherein the solid bacterial agent further comprises an inorganic nutrient comprising N, P₂O₅ and K₂O in a mass ratio of 4-8:1-3:1-3, and a mass of the inorganic nutrient is 4 to 9 times a mass of the Bacillus subtilis BS40-4 in the form of powders.

6. The bacterial agent of claim 4, wherein the solid bacterial agent further comprises an inorganic nutrient comprising N, P₂O₅ and K₂O in a mass ratio of 4-8:1-3:1-3, and a mass of the inorganic nutrient is 4 to 9 times a mass of the Bacillus subtilis BS40-4 in the form of powders.

7. The bacterial agent of claim 2, wherein:
the bacterial agent is a liquid bacterial agent;
the auxiliary agent comprises a nutrient solution; and
the nutrient solution comprises an inorganic nutrient comprising N, P₂O₅ and K₂O in a mass ratio of 4-8:1-3:1-3, and a mass concentration of the inorganic nutrient is 10-20% in the liquid bacterial agent.

8. The bacterial agent of claim 7, wherein the Bacillus subtilis BS40-4 strain is obtained by filtration of a Bacillus subtilis BS40-4 fermentation broth through a plate-and-frame filter.

9. The bacterial agent of claim 7, wherein a number of viable Bacillus subtilis BS40-4 in the liquid bacterial agent is 1×10⁸ CFU/mL to 5×10¹⁰ CFU/mL.

10. The bacterial agent of claim 8, wherein a number of viable Bacillus subtilis BS40-4 in the liquid bacterial agent is 1×10⁸ CFU/mL to 5×10¹⁰ CFU/mL.

11. A method for composting organic wastes, the method comprising culturing the bacterial agent of claim 2 in the organic wastes.

12. The method of claim 11, comprising pulverizing the organic wastes, hydrolyzing pulverized organic wastes at 70 to 100° C., adding the bacterial agent accounting for 0.1-0.3 wt. % of the organic wastes to the pulverized organic wastes, and evenly mixing for fermentation.

13. The method of claim 12, wherein:
a water content in the organic wastes is 50 to 70 wt. %;
a hydrolysis time is 2 to 10 hours; and
intermittent aeration is performed during a fermentation process of the organic wastes, and the intermittent aeration comprises conducting continuous aeration for 30 to 120 minutes and then stopping the aeration for 30 to 40 minutes; an aeration rate per cubic meter of the pulverized raw material is 50 to 200 L/min during the continuous aeration.
